(19) United States
(12) Patent Application Publication
(10) Pub. No.: US 2011/0217345 A1
(43) Pub. Date: Sep. 8, 2011

(54) EMULSION COMPOSITION, METHODS OF SOFTENING FIBROUS STRUCTURES USING THE SAME, AND FIBROUS SUBSTRATE TREATED THEREWITH

(76) Inventors: Fuming Huang, Shanghai (CN); Junjie Shen, Shanghai (CN)

(21) Appl. No.: 13/055,072

(22) PCT Filed: Jul. 17, 2009
(86) PCT No.: PCT/CN2009/072806

§ 371 Date: May 18, 2011

(30) Foreign Application Priority Data
Jul. 22, 2008 (CN) 200810040819.5

(51) Int. Cl. C09D 7/12; C11D 3/60; A61K 8/97; A61K 8/02; A61Q 19/00; B32B 29/00

(52) U.S. Cl. 424/401; 510/527; 424/744; 106/287.13; 428/447

(57) ABSTRACT

An emulsion composition, methods of softening fibrous structures using the same, and fibrous substrate treated therewith. The composition comprising: (A) 0.01 to less than 25.0 parts by weight of a diorganopolysiloxane; (B) 0.01 to less than 20.0 parts by weight of softening agents; (C) 8.0 to 60.0 parts by weight of water; and (D) 20.0 to 90.0 parts by weight of water-soluble monovalent or polyvalent alcohols, wherein the continuous phase of said emulsion composition is an aqueous solution consisting of said (C) water and said (D) water-soluble monovalent or polyvalent alcohols in the ratio of from 1:0.5 to 1:10, and total content of said component (C) and (D) is in the rage of 30.0 and 98.0 parts by weight of said emulsion composition.

### OBJECT OF INVENTION

This invention relates to a new emulsion composition having the continuous phase of an aqueous solution consisting of water and water-soluble alcohols. This new emulsion composition is further characterized by comprising a diorganopolysiloxane as emulsion particle emulsified in the said aqueous solution, and a softening agent. This emulsion composition keeps good temporal stability and homogeneous appearance for months in the storage.

This invention also relates to a new softening emulsion composition for a fibrous substrate, especially for a tissue paper. This softening emulsion composition provides not only softness feeling, but also excellent slippery, moisture/lotion feeling, and comfortable fluffiness to human skin for the fibrous substrate treated therewith. In addition, the specific water absorbency of the fibrous substrate is maintained after treating by the softening emulsion composition.

The present invention also relates to a method of softening fibrous structure using said emulsion composition, a fibrous substrate treated therewith and especially relates to a tissue paper product. The tissue paper treated with the emulsion composition of the present invention has an enhanced softness, slippery, moisture/lotion feeling and fluffy feeling to human skin through incorporation of an effective amount of softening agent, diorganopolysiloxane and water-soluble alcohols, especially glycerin, applied to dry-end tissue paper webs without desiccation process. The prepared tissue paper product keeps its specific water absorbency from fibrous paper structures without any destroy by treating process.

### BACKGROUND ART

For softening tissue paper product, some functional silicones are applied to the tissue product. In such application, some softening agents and some alcohols like glycerin are also applied to the tissue product.

For example, PCT Patent Publication WO1996/024719 [“SOFT TISSUE PAPER CONTAINING AN OIL AND A POLYHYDROXY COMPOUND” (the procter & gamble corporation)] disclosed the tissue paper having an enhanced bulk and tactile softness through incorporation of an effective amount of a polyhydroxy compound and an oil applied to wet tissue paper webs. The add-on of a polyhydroxy compound and an oil on the said tissue paper were both from 0.01%-5% on a dry fiber weight basis. The said polyhydroxy compound and oil include glycerin and polysiloxane based oil, respectively.

Japan Patent publication H(heisei)07-145596 [“COMPOSITION FOR TREATING WIPING PAPER” (Dow Corning Toray silicone Co., Ltd.)] described a treatment composition for wiping paper which was composed of a specific diorganosiloxane and a polyhydric alcohol and can be used most suitably for giving tissue paper the flexibility, slipperiness and soft touch. This patent publication also mentioned the constituent was emulsified with a surfactant to prepare an emulsion with more than 50 wt % of water. The treatment composition was applied to dry tissue paper with a desiccation process, and the range of applied amount of this composition was 0.2-5.0% of the weight.

These softening compositions and softening methods are applied to many tissue paper products having improved softness and feelings. However, these existing technologies are not necessarily satisfactory for the reason that the softness of tissue paper product treated therewith is not enough to feel good to human skin in daily use and that the provided feeling of the prepared tissue paper is mainly involved to its softness.

Furthermore, the present inventors found another problem for such softening compositions and softening methods, except for its insufficient feeling of prepared tissue product. The problem is that some properties like water absorbency from fibrous paper structures by its nature could be destroyed by the softening treatment or desiccation process using the existing softening compositions like oil-in-water emulsions or add-on glycerin process. That is to say, inventors found that some natural and advantageous properties of fibrous substrates could be spoiled in exchange for providing some softness to the substrates in existing technologies.

### SUMMARY OF THE INVENTION

The present invention was pursued in order to solve the problems cited above and inventors developed a new emulsion composition comprises (A) a diorganopolysiloxane, (B) a softening agent, (C) water and (D) water-soluble monovalent or polyvalent alcohols, wherein the continuous phase of said emulsion composition is an aqueous solution consisting of said (C) water and said (D) water-soluble monovalent or polyvalent alcohols in the ratio of from 1:0.5 to 1:10, and total content of said component (C) and (D) is in the rage of 30.0 and 98.0 parts by weight of said emulsion composition. Inventors also developed a new softening emulsion composition comprising thereof, and a method of softening fibrous structure using said emulsion composition, a fibrous substrate treated therewith and especially a tissue paper product treated therewith.

Specifically, the present invention relates to an emulsion composition comprising:

(A) [0.01 to less than 25.0 parts by weight of] a diorganopolysiloxane;
(B) [0.01 to less than 20.0 parts by weight of] a softening agent;
(C) [8.0 to 60.0 parts by weight of] water; and
(D) [20.0 to 90.0 parts by weight of] water-soluble monovalent or polyvalent alcohols
wherein the continuous phase of said emulsion composition is an aqueous solution consisting of said (C) water and said (D) water-soluble monovalent or polyvalent alcohols in the ratio of from 1:0.5 to 1:10, and total content of said component (C) and (D) is in the rage of 30.0 and 98.0 parts by weight of said emulsion composition.

This emulsion composition keeps good temporal stability and homogeneous appearance for months in the storage. And, this emulsion composition provides not only softness feeling, but also excellent slippery, moisture/lotion feeling, and comfortable fluffiness to the fibrous substrate treated therewith. The natural and advantageous properties like water absorbency are maintained after treating the fibrous substrates therewith.

Additionally, another desiccation process and add-on glycerin process for manufacturing the tissue product treated therewith can be skipped, for the continuous phase of this emulsion composition consists essentially of aqueous alcohol solution, preferably glycerin solution. By using this emulsion composition, diorganopolysiloxanes and alcohols applied to dry-end tissue paper webs without desiccation process.

### DISCLOSURE OF INVENTION

The objects cited above can be achieved by the following.

{1} An emulsion composition comprising:
(A) [0.01 to less than 25.0 parts by weight of] a diorganopolysiloxane;
(B) [0.01 to less than 20.0 parts by weight of] a softening agent;
(C) [8.0 to 60.0 parts by weight of] water; and
(D) [20.0 to 90.0 parts by weight of] water-soluble monovalent or polyvalent alcohols
wherein the continuous phase of said emulsion composition is an aqueous solution consisting of said (C) water and said (D) water-soluble monovalent or polyvalent alcohols in the ratio of from 1:0.5 to 1:10, and total content of said component (C) and (D) is in the rage of 30.0 and 98.0 parts by weight of said emulsion composition.

{2} The emulsion composition according to {1}, wherein said continuous phase of said emulsion composition has a Glycerin content of from 30 to 90 parts by weight.

{3} The emulsion composition according to {1} or {2}, wherein the emulsion particle comprising said component (A) has a mean particle size of 50 to 10,000 nanometers.

{4} The emulsion composition according to any of {1} to {3}, wherein the emulsion particle comprising said component (A) of a mean particle size of 50 to 300 nanometers.

{5} The emulsion composition according to any of {1} to {4}, wherein the emulsion particle comprising said component (A) is a diorganopolysiloxane having at least one group selecting from the group consisting of amino-functional group, polyoxyalkylene group, sulfonate-functional group and epoxy-functional group in the molecule.

{6} The emulsion composition according to any of {1} to {5}, further comprises (E) [0.01 to less than 20.0 parts by weight of] a moisturizing agent and optionally, (F) preservatives.

{7} A softening emulsion composition for a fibrous substrate comprises the emulsion composition according to any of {1} to {6}.

{8} The softening emulsion composition for a fibrous substrate according to {7}, wherein said fibrous substrate is selected from the group consisting of wipes, fabrics, textiles, and papers.

{9} A method of softening fibrous structures by treating substrate having fibrous structure with the emulsion composition according to any of {1} to {6}.

{10} A fibrous substrate treated with the emulsion composition according to any of claims {1} to {6}.

{11} The fibrous substrate according to {10}, said fibrous substrate is a tissue paper product.

After this, the components of this invention, other optional components, emulsion composition itself and its preparation are explained specifically.

### [Component (A)]

Component (A) is a diorganopolysiloxane including amino functional polysiloxanes, epoxy functional polysiloxanes sulfonate functional polysiloxanes and polyoxyalkylene functional polysiloxanes. Different functional groups play an important role in providing enhanced softening property to polysiloxane structure itself and polysiloxane backbone provides ultra slippery. Furthermore, comfortable fluffiness is achieved by the combination between component (A) and component (D) in the emulsion state.

More precisely, the diorganopolysiloxane is represented by the following average structural formula (1):

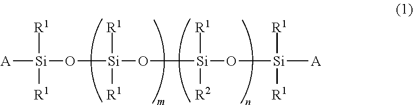

wherein R¹ is an unsubstituted, fluoro-, alkoxy- or hydroxyl-substituted monovalent hydrocarbon group. The unsubstituted monovalent hydrocarbon group is exemplified by methyl, ethyl, propyl, or similar alkyl groups; phenyl, tolyl, xylyl, or similar aryl groups; or aralkyl groups. The fluoro-substituted monovalent hydrocarbon group is exemplified by 3,3,3-trifluoropropyl, 3,3,4,4,4-pentafluorobutyl, or similar perfluoroalkyl groups. Among the above, R¹ is preferably the unsubstituted monovalent hydrocarbon group, preferably the alkyl group or aryl group, and, in particular, methyl or phenyl group. A part of groups represented by R¹ bonded to silicon atoms, especially a part of groups represented by R¹ on the molecular terminals, may be replaced by hydroxyl group or alkoxy groups, such as methoxy, ethoxy, or propoxy groups.

R² is a functional group except for R¹ and play an important role in providing enhanced softening property to polysiloxane structure itself and polysiloxane backbone provides ultra slippery. This functional group is exemplified by polyoxyalkylene group represented by the following formula (2): —R³—O—(C₂H₄O)ₐ (C₃H₆O)_{b}R⁴ [where R³ is an alkylene group with 2 to 30 carbon atoms; R⁴ is a group selected from hydrogen atom, an alkyl group with 1 to 30 carbon atoms, or an organic group of the following formula: —(OC)—R⁵ (where R⁵ is an alkyl group with 1 to 30 carbon atoms), 1≦a+b≦30]; methoxycarbonylpropyl, ethoxycarbonylpropyl, or similar alkoxycarbonylalkyl groups; acetoxypropyl, propionoxypropyl, or similar alkylcarbonyloxyalkyl groups; 3-aminopropyl, 3-(aminoethyl)aminopropyl, ethanediamino-2-methylpropyl- or alkylaminocarbonylalkyl-, alkylcarbonylaminoalkyl-, OH-terminated aminoethylaminoisobutylmethyl-[ex. —CH2CH(CH3)CH2—N(CH2CH(OH)CH2OH)—CH₂—NH(CH2CH(OH)CH2OH)] or similar amino-functional groups; epoxy group; carboxyl group; glyceryl group; polyglyceryl group; saccharide group; polysaccharide group; mercapto group; carbinol group; sulfonate group and amide group. In particular, the preferred diorganopolysiloxane is the methylpolysiloxanes having amino-functional groups, polyoxyalkylene groups, sulfonate-functional groups or epoxy-functional groups.

Among them, most preferred group is amino-functional group represented by the following formula: —(CH₂)_{d}—(NHCH₂CH₂)ₑ—NH—R⁴ or —C_{d}H_{2d}-(NHCH(OH)CH(OH))ₑ—NH—CH(OH)CH(OH) (d ranges from 1 to 10; e ranges from 0 to 10).

In the above formula, “m” and “n” preferably satisfy the following conditions: 0≦m; 0≦n and 0≦m+n≦5000; but when n=0, at least one “A” is R². It is preferable that “m” ranges from 5 to 2000, and “n” ranges from 0 to 100.

There are no special restrictions with regard to viscosity of component (A); however, from the viewpoint of preparing emulsion with improved temporal stability, it is recommended that the viscosity of component (A) at 25° C. ranges preferably from 5 to 1,000,000 mPas at 25 degrees C.

Optionally, (A) a diorganopolysiloxane can be combined with other dimethylpolysiloxane. Such dimethylpolysiloxane can be exemplified by linear-chained dimethylpolysiloxanes, branched methylpolysiloxanes, volatile cyclic dimethylpolysiloxanes; dimethylpolysiloxane capped at both molecular terminals with trimethylsiloxy groups, methylphenylpolysiloxane having both molecular terminals capped with trimethylsiloxy groups, dimethylpolysiloxane capped at both molecular terminals with silanol(OH) groups, which have a viscosity at 25° C. in the range of 0.65 to 10,000 mm2/s. Such dimethylpolysiloxanes are known softening agents and can be included in below component (B).

### [Component (B)]

Component (B) is a softening agent. As used herein, the term “softening agent” refers to any chemical ingredient which improves the tactile sensation (as feeling to human skin) perceived by the consumer who holds a particular paper product and rubs it across the skin. Although somewhat desirable for towel products, softness is a particularly important property for facial and toilet tissue paper products. Such tactilely perceivable softness can be characterized by, but is not limited to, friction, flexibility, and smoothness, as well as subjective descriptors, such as a feeling like lubricious, velvet, silk or flannel. Suitable materials include those which impart a lubricious feel to tissue paper.

Such component is exemplified as aforementioned dimethylpolysiloxanes, basic waxes such as paraffin and beeswax and oils such as mineral oil as well as petrolatum and more complex lubricants and quaternary ammonium compounds, fluorocarbons, substituted C10-C22 alkanes, substituted C10-C22 alkenes, in particular ester derivatives of fatty alcohols and fatty acids (such as fatty acid amides, fatty acid condensates and fatty alcohol condensates), sugar derivatives (such as ethers and esters) and mixtures thereof.

In this invention, preferred softening agents are quaternary ammonium compounds; mono-, di-, or triester quaternary ammonium compounds; di-quaternary esterified ammonium compounds, or mixtures thereof. The quaternary ammonium compounds may have long (C10-C22) alkyl chains Such quaternary ammonium compound may be an imidazolinium compound, such as an imidazolinium salt or mixtures thereof.

Examples of suitable quaternary ammonium compounds may be selected from compounds having the general formula

(R¹)_{4-b}—N⁺—(R²)_{b}X⁻

wherein R¹ is a C1 to C6 alkyl group, R² is a C10 to C22 alkyl group, b is an integer from 1 to 3. Other similar compounds may include the monoester, diester, moncamide, and diamide derivatives of the simple quaternary ammonium salts. X can be any quaternary ammonium-compatible anion, for example, acetate, chloride, bromide, methyl sulfate, formate, sulfate, nitrate and the like can also be used in the present invention.

Other quaternary ammonium compounds include the well-known dialkyldimethylammonium salts (e.g., ditallowedimethylammonium chloride, ditallowedimethylammonium methyl sulfate, di(hydrogenated tallow)dimethyl ammonium chloride, etc.) and trialkylmethylammonium salts (e.g., tritallowmethylammonium chloride, tritallowmethylammonium methyl sulfate, tri(hydrogenated tallow)methyl ammonium chloride, etc.).

Examples of such quaternary ammonium compounds include alkyltrimethyl ammonium chloride, stearyltrimethyl ammonium chloride, lauryltrimethyl ammonium chloride, cetyltrimethyl ammonium chloride, beef tallow alkyltrimethyl ammonium chloride, behenyltrimethyl ammonium chloride, octyltrimethyl ammonium hydroxide, dodecyltrimethyl ammonium hydroxide, stearyltrimethyl ammonium bromide, behenyltrimethyl ammonium bromide, distearyldimethyl ammonium chloride, dicocoyldimethyl ammonium chloride, dioctyldimethyl ammonium chloride, di-(POE) oleylmethyl ammonium chloride (2EO), benzalkonium chloride, alkylbenzalkonium chloride, alkyldimethylbenzalkonium chloride, benzethonium chloride, stearyldimethylbenzyl ammonium chloride, lanolin-derived quaternary ammonium salts, stearic acid diethylaminoethylamide, stearic acid dimethylaminopropylamide, behenic acid amide propyldimethyl hydroxypropyl ammonium chloride, cetylpyridinium chloride, tall oil alkylbenzylhydroxyethyl imidazolinium chloride, or benzyl ammonium salts.

### [Component (C)]

Component (C) is water. The water should not contain components harmful to humans and should be clean. Water may be exemplified by tap water, purified water, and mineral water.

### [Component (D)]

Component (D), water-soluble monovalent or polyvalent alcohols are a liquid that is characterized by excellent compatibility with water at room temperature. This component provides moisture/lotion feeling to the fibrous substrates and comfortable fluffiness by the combination with (A) diorganopolysiloxane in the emulsion state.

Such component (D) may be one alcohol or a mixture of water-soluble monovalent or polyvalent alcohols. The preferable water-soluble alcohol is monovalent or polyvalent alcohol having 2 to 8 carbon atoms. Furthermore, preferred component (D) is hypoallergenic alcohols or mixture thereof, which does not cause irritation or minimal irritation on human skin when remained on the surface of tissue paper products.

Specific examples of such component (D) include ethanol, n-propanol, isopropanol, n-butanol, or a similar monovalent lower alcohol; 1,3-butyleneglicol, ethyleneglycol, propyleneglycol, or a similar bivalent alcohol; polyethyleneglycol, dipropyleneglycol, polypropyleneglycol, or a similar polyalkyleneglycol; glycerin, diglycerin, trimethylolpropane, pentaerythritol, sorbitol, gluconic acid represented by the formula: HO—CH₂(CH(OH)₄—COOH or a similar polyalcohol.

As a softening emulsion for a tissue paper treatment, most preferable component (D) is glycerin or mixture thereof, and preferable glycerin content is from 30 to 90 parts by weight in this emulsion composition.

### [Continuous Phase of the Emulsion]

This inventive emulsion composition is characterized in that the continuous phase of said emulsion composition is an aqueous solution consisting of (C) water and (D) water-soluble monovalent or polyvalent alcohols in the ratio of from 1:0.5 to 1:10, and total content of said component (C) and (D) is in the rage of 30.0 and 98.0 parts by weight of said emulsion composition.

Although this inventive emulsion composition has analogous emulsification state to oil-in-water emulsion, the emulsion particle comprising (A) diorganopolysiloxane is emulsified into the aqueous solution based on (D) water-soluble monovalent or polyvalent alcohols.

Depending on its solubility into the aqueous solution, (B) softening agent may be dispersed in the aqueous solution (continuous phase having hydrophilicity), in the emulsion particle (oily particle phase having hydrophobicity) or its emulsion interface as surfactant. Anyway, the emulsification state of this invention is so stable that this emulsion composition keeps good temporal stability and homogeneous appearance for months in the storage.

The respective softening effects of (A) diorganopolysiloxane, (B) softening agent and (D) water-soluble monovalent or polyvalent alcohols are further improved in this specific emulsified state, and not only softness feeling, but also excellent slippery, moisture/lotion feeling, and comfortable fluffiness to human skin are achieved in the fibrous substrate treated therewith. In addition, the high content of (D) water-soluble monovalent or polyvalent alcohols, which speeds the evaporation of (C) water, makes no need for the desiccation process for the tissue treating process. For this, the specific water absorbency of the fibrous substrate is maintained after treating by this emulsion composition.

As a softening emulsion composition, most preferred continuous phase of this emulsion composition has a glycerin content of from 30 to 90 parts by weight in the total weight pts of emulsion composition. Then, glycerin may be combined with other hypoallergenic alcohols or mixture thereof.

As a softening emulsion composition, the preferred ratio of (C) water and (D) water-soluble monovalent or polyvalent alcohols is from 1:0.75 to 1:8, more preferred one is from 1:1 to 1:5 in the continuous phase of this emulsion.

Additionally, if a softening composition contains large amount of component (D) water-soluble alcohols, in particular, ethanol, glycerin or a similar lower alcohols, such composition would be highly flammable for these alcohols.

However, in this softening emulsion composition, aqueous solution with (C) water reduces flammability and provides higher safety during manufacturing, storage, and transportation. Such low flammability makes this inventive emulsion composition easy-to-use and safe softening composition especially for treating tissue paper products.

Furthermore, water-soluble components, such as water-soluble ionic surfactants, aqueous perfumes, aqueous pigments or emollients like aqueous plant extracts can be preliminarily dispersed in (C) water and then compounded with the composition for the preparation of the emulsion composition of the present invention.

On the other hand, for the aqueous solution of (C) water and (D) water-soluble alcohols has much lower freezing point than water itself, this emulsion composition is difficult to be frozen in winter of the cold region and provides good freeze/thaw stability.

### [Emulsion Particle]

The emulsion particle of this emulsion composition comprises said (A) diorganopolysiloxane and other hydrophobic components compatible with (A) diorganopolysiloxane. When some or all parts of said (B) softening agent is hydrophobic, such (B) softening agent may be comprised in the emulsion particle or on the emulsion interface as surfactant.

There are no particular limitations concerning the emulsion particle size, but it is preferable that when the emulsion composition of the present invention is prepared as a softening emulsion composition for fibrous substrates, it should have average emulsion particles not exceeding 10.0 μm, when measured by the laser diffraction/dispersion method or Coulter Counter Method. To keep its temporal stability for months without phase separation, the mean particle size should range between 50 nanometers and 10.0 μm (i.e. 10,000 nanometers).

Furthermore, when the emulsion composition of the present invention is prepared as a softening emulsion composition for tissue paper products, preferred average emulsion particles less than 0.5 μm (500 nanometers), and in particular less than 0.3 μm (300 nanometers), when measured by the laser diffraction/dispersion method or Coulter Counter Method.

Most preferred emulsion composition is a micro-emulsion having a particle size of 50 to 300 nanometers, especially around 150 nanometers. Such preferred micro-emulsion composition has a hazy, semi-transparent or transparent appearance, and keeps its homogeneous emulsification state for months in the storage.

### [Emulsification Procedure]

The emulsification procedure is carried out using standard emulsion mechanical shear equipments or operations. For instance, such mixing and emulsification can be carried out by using any suitable source of shear, such as a high speed stirrer, a homogenizer, sonolator, micro-fluidizer, Turello change can mixer, Ross mixer, Eppenbach colloid mill, homomic line mils, dynamic mixers, rotary disc mixers, twin screw extruders and similar devices. Any named mechanical shear devices can be adopted in stages of manufacturing and two or more steps of different emulsification procedures can be adopted to prepare the emulsion composition having desirable particle sizes. Where appropriate, hand mixing can also be employed.

There are no particular limitations concerning the manufacturing steps for the emulsification procedure, for instance, the emulsion composition of the present invention comprising above component (A), component (B), component (C) and component (D) can be prepared in accordance with the following steps.

(1) A predetermined amount of (A) a diorganopolysiloxane, a predetermined amount of (B) a softening agent, and optionally a predetermined amount of (G) other surfactants are placed in a high-speed stirring mixer and mixed by stirring for a predetermined time.
(2) Predetermined amounts of (C) water, optionally (E) a moisturizing agent, (F) preservatives, (H) pH adjusting agent are placed in a high-speed stirring mixer and mixed together by stirring for a predetermined time. In this step, predetermined amounts of (C) water can be gradually added in stages and mixed therewith by stirring.
(3) Predetermined amounts of (D) water-soluble monovalent or polyvalent alcohols are placed in a high-speed stirring mixer and mixed together by stirring for a predetermined time until the all components are dispersed and emulsified in homogeneous state.

The high-speed stirring mixer used here is exemplified by Homomixers, paddle mixers, Henschel mixers, Homodispersers, colloid mills, stirring mixers for vacuum mixing, twin screw extruders, etc. However, there are no particular limitations concerning the type of the mixer as long as it has superior emulsifying power and produces stable emulsions.

In any step (2) or (3), (C) water can be replaced with some mixture thereof or aqueous solution consisting of component (C) and component (D). Such pre-mixing of components can be adopted in response to specific equipments or manufacturing process in practice.

### [Component (G): Surfactants]

In this invention, some component (B) also plays a part of surfactant to emulsify component (A) and other hydrophobic components compatible therewith into the aqueous solution of component (C) and (D). That is to say, some part of component (B) has overlapped function between a softening agent and a surfactant.

However, when component (B) has poor emulsification capacity and/or there is a intention to improve the temporal stability of emulsion, the emulsion composition of the present invention can further contain (G) surfactants. Preferred amount of (G) surfactants is 0.5 to 20.0 parts by weight of said (A) a diorganopolysiloxane. When (G) surfactants comprises nonionic surfactants, more preferred amount of (G) surfactants is 1.0 to 10.0 parts by weight of said (A) a diorganopolysiloxane.

There are no particular limitations with regard to aforementioned (G) surfactants of one or more types. Examples of this surfactant include an ionic surfactant, nonionic surfactant, or a combination of both. From the viewpoint of improved temporal stability of the emulsion composition, it is preferable to use (G1) a nonionic surfactant of one or more types.

When emulsion compositions are prepared, typically the low HLB nonionic surfactant may be first added to a preformed mixture of component (A) and other hydrophobic components compatible therewith. Then an aqueous solution containing the high HLB surfactant is prepared, to which is admixed the aforementioned mixture. The combined components are mixed to form a crude emulsion, which is then emulsified to complete the emulsion composition.

More specifically, examples of the nonionic surfactants (G1) of one or more types is exemplified by polyoxyalkylene ethers, polyoxyalkylene alkyl ethers, polyoxyalkylene fatty acid esters, polyoxyalkylene fatty acid diesters, polyoxyalkylene resinic acid esters, polyoxyalkylene (hydrogenated) castor oils, polyoxyalkylene alkyl phenols, polyoxyalkylene alkyl phenyl ethers, polyoxyalkylene phenyl phenyl ethers, polyoxyalkylene alkyl esters, sorbitan fatty acid esters, polyoxyalkylene sorbitan alkyl esters, polyoxyalkylene sorbitan fatty acid esters, polyoxyalkylene sorbitol fatty acid esters, polyoxyalkylene glycerin fatty acid esters, polyglycerol alkyl ethers, polyglycerol fatty acid esters, sucrose fatty acid esters,

fatty acid alkanolamides, alkylglucosides, polyoxyalkylene fatty acid bisphenyl ethers, polypropylene glycols, perfluoropolyether-type surfactants, polyoxyethylene-polyoxypropylene block copolymers, and alkyl polyoxyethylene-polyoxypropylene block copolymer ethers.

Examples of the ionic surfactant (G2) of one or more types include an anionic surfactant, cationic surfactant, or amphoteric surfactant. Examples of the anionic surfactant include saturated or unsaturated fatty acid salts (e.g., sodium laurate, sodium stearate, sodium oleate, and sodium linoleate, etc.), long-chain alkylsulfuric acid salts, alkylbenzene sulfonic acids (e.g., hexylbenzene sulfonic acid, octylbenzene sulfonic acid, and dodecylbenzene sulfonic acid, etc.) and their salts, polyoxyalkylene alkyl ether sulfates, polyoxyalkylene alkenyl ether sulfates, polyoxyethylene alkyl sulfate ester salts, sulfosuccinic acid alkyl ester salts, polyoxyalkylene sulfosuccinic acid salts, polyoxyalkylene sulfosuccinic acid ester salts, alkali-metal salt of sulfosuccinic acid ester of polyoxyalkylene-modified dimethylpolysiloxane, polyoxyalkylene alkyl phenyl ether sulfates, long-chain alkanesulfonic acid salts, long-chain alkylsulfonate, polyoxyethylene alkyl phenyl ethyl sulfates, polyoxyalkylene alkyl ether acetates, long-chain alkylphosphates, polyoxyalkylene alkyl ether phosphates, acylglutamic acid salts, a acylsulfonic acid salts, long-chain alkylsulfonic acid salts, alkylallyl sulfonic acid salts, long-chain α-olefin sulfonates, alkylnaphthalenesulfonic acid salts, long-chain alkane sulfonic acid salts, long-chain alkyl or alkenylsulfonic acid salts, long-chain alkylamidesulfonic acid salts, long-chain alkyl or alkenylphosphoric acid salts, alkylamide phosphates, alkyloylalkyltaurine salts, N-acylaminoacid salts, sulfosuccinic acid salts, alkylalkyl ether carboxylic acid salts, amide ether carboxylates, a-sulfo fatty acid ester salts, alanine derivatives, glycine derivatives, or arginine derivatives. Examples of the aforementioned include sodium salts, potassium salts, or similar alkali metal salts; triethanolamine salts or similar alkanolamine salts, as well as ammonium salts, but sodium salts are preferable,

Examples of the amphoteric surfactants (G3) include phospholipids, such as lecithins, phosphatidyl ethanolamine, phosphatidic acid, phosphatidyl inositol, phosphatidyl serine, phosphatidyl choline, phosphatidyl glycerol, sphingomyelin, cardiolipin, or hydrogenates of the above compounds. Most preferable are hydrogenated soybean lecithin, egg yoke lecithin, rape lecithin, or a similar hydrogenated natural lecithin.

### [(E) Moisturizing Additive]

To improve slippery and moisture/lotion feeling to the fibrous substrate treated therewith, this inventive emulsion composition preferably comprises (E) Moisturizing additive of one or more types. In cases, such components may be called as “emollients”.

Moisturizing additives have the ability to attract water vapor (moisture) from the atmosphere and bring it to the surface of treated substrate, which results in slippery and moisture/lotion feeling and alleviation of dryness.

Examples of such component (E) include urea, sodium lactate, polysaccharides, saccharlde isomerate, sorbltol, urea, sodium PCA, plant extract and other biological actives like. Preferred component (E) is sodium lactate, natural vegetable extracts, seaweed extracts, and herbal medicines. Among them, aloe vera extract and sodium lactate are particularly preferable.

Examples of other plant extracts and other biological actives are as follow: Angelica keiskei extract, avocado extract, Hydrangea serrata extract, Althea extract, Arnica extract, aloe extract, apricot extract, apricot kernel extract, Gingko biloba extract, Fennel fruit extract, Turmeric root extract, Oolong tea extract, Rosa multiflora extract, Echinacea angustifolia leaf extract, Scutellaria baicalensis root Extract, Phellodendron amurense extract, Coptis rhizome extract, Hordeum vulgare seed extract, Hypericum perforatum extract, Lamium album extract, Nasturtium officinale extract, orange extract, dried sea water solution, seaweed extract, hydrolyzed elastin, hydrolyzed wheat fines, hydrolyzed silk, Chamomile extract, carrot extract, Artemisia capillaris flower extract, Licorice extract, Karkade extract, Pyracantha fortuneana extract, kiwi extract, Cinchona extract, cucumber extract, guanosine, Gardenia florida extact, Sasa veitchii extract, Sophora angustifolia extract, walnut extract, grapefruit extract, Clematis vitalba leaf extract, chlorella extract, Morus alba root extract, Gentiana lutea extract, black tea extract, yeast extract, burdock extract, fermented rice bran extract, rice germ oil, Comfrey extract, collagen, Vaccinum vitis idaea extract, Asiasarum root extract, Bupleurum falcatum extract, umbilical extract, Salvia extract, Soapwort extract, Sasa bamboo grass extract, Crataegus cuneata fruit extract, Zanthoxylum piperitum extract, Shiitake extract, Rehmannia root extract, Lithospermum erythrorhizone root extract, Perilla ocymoides extract, Tilia cordata flower extract, Spiraea ulmaria extract, Paeonia albiflora extract, Acorus calamus root extract, Betula alba extract, Equisetum arvense extract, Hedera helix extract, Crataegus oxyacantha extract, Sambucus nigra extract, Achillea millefolium extract, Mentha piperita leaf extract, Sage extract, Malva sylvestris extract, Cnidium officinale root extract, Swertia japonica extract, Soybean extract, Zizyphus jujuba fruit extract, thyme extract, tea extract, Eugenia caryophyllus flower extract, Imperata cylindrica extract, Citrus unshiu Marc extract, Angelica root extract, Calendula officinalis extract, Prunus persica stone extract, Citrus aurantium peel extract, Houttuynia cordata extract, tomato extract, natto extract, carrot extract, garlic extract, Rosa canina fruit extract, Hibiscus extract, Ophiopogon extract, Nelumbo nucifera extract, parsley extract, honey, Witch hazel extract, Parietaria officinalis extract, Isodon trichocarpus extract, bisabolol, Eriobotrya japonica extract, Coltsfoot flower extract, Petasites japonicus extract, Poria cocos extract, Butcher's broom extract, grape extract, propolis, Luffa cylindrica fruit extract, Safflower flower extract, peppermint extract, Tillia miquellana extract, Paeonia suffruticosa root extract, hops extract, Pinus sylvestris cone extract, horse chestnut extract, Japanese skunk-cabbage extract, Sapindus mukurossi peel extract, Melissa extract, peach extract, Centaurea cyanus flower extract, Eucalyptus extract, Saxifraga sarementosa extract, Citrus junos extract, Coix seed extract, Artemisia princeps extract, lavender extract, apple extract, lettuce extract, lemon extract, Astragalus sinicus extract, rose extract, rosemary extract, Roman chamomile extract, and royal jelly extract.

### [(F) Preservatives]

Component (F) is preservatives, that is normally added to emulsion compositions and that can be added to the emulsion of the present invention is exemplified by a group of (F1) antiseptics including paraoxybenzoic acid alkyl ester, benzoic acid, sodium benzoate, sorbic acid, potassium sorbate, phenoxyethanol and by a group of (F2) antibacterial agent including benzoic acid, salicylic acid, carbolic acid, sorbic acid, paraoxybenzoic acid alkyl esters, p-chlormethacresol, hexachlorophene, benzalkonium chloride, chlorhexidine chloride, trichlorocarbanilide, trichlosan, light-sensitive ingredients, phenoxyethanol, methylisothiazolinone, etc.

These compounds should be used in amounts sufficient to prevent decay of the emulsion composition.

### [Optional Components]

The emulsion composition of may further contain (H) pH adjuster, (J) fragrance (K) an antioxidant, (L) a water-soluble polymer or other (M) biologically active component which are described below, or at least one types of these agents within the limits that are not harmful to the properties thereof.

(H) pH adjuster, that is normally added to emulsions and that can be added to the emulsion composition of the present invention is exemplified by lactic acid, citric acid, glycolic acid, succinic acid, tartaric acid, dl-malic acid, potassium carbonate, sodium hydrogencarbonate, ammonium hydrogencarbonate, etc.

(J) fragrance, that is normally added to daily using fibrous substrates and that can be added to the emulsion composition of the present invention is used for imparting to the treated substrates a certain aroma, scent, or for masking unpleasant odor. There are no special restrictions with regard to the type of fragrance, provided that it is a conventional fragrance normally added to tissue paper products, and examples of this component include various extracts shown above as biologically active components; extracts from flowers, seeds, leaves, and roots of various plants; fragrances extracted from seaweeds; fragrances extracted from various parts or secretion glands of animals (e.g., musk and sperm oil), or artificially synthesized fragrances (e.g., menthol, musk, ethyl acetate, or vanila).

(K) antioxidant, that is normally added to emulsions and that can be added to the emulsion composition of the present invention is exemplified by tocopherol, butylated hydroxyanisole, dibutylhydroxytoluene, phytic acid, carotenoid, flavonoid, tannin, lignan, or saponin. This agent should be added in an amount sufficient to protect the components in the emulsion from oxidation.

(L) water-soluble high molecular polymer and water-swellable mineral clay, that are used for increasing viscosity, improving temporal stability, and enhancing feel of use of the fibrous substrates treated therewith. It is preferable that the water-soluble polymer and water-swellable mineral clay are dissolved or dispersed in water to prepare a uniform aqueous solution or dispersion, and then the prepared solution or dispersion is mixed with other components. The water-soluble polymer may be of an amphoteric, cationic, anionic, or a nonionic type. The water-soluble polymers and water-swellable mineral clay can be used together, or two or more water-soluble polymers can be used in combination.

Examples of (M) other biologically active component are as follow: deoxyribonucleic acid, mucopolysaccharides, sodium hyaluronate, sodium chondroitin sulfate, collagen, elastin, chitin, chitosan, hydrolyzed eggshell membrane and other biopolymers etc.; glycine, valine, leucine, isoleucine, serine, threonine, phenylalanine, arginine, lysine, aspartic acid, glutamate, cystine, cysteine, methionine, tryptophan and other amino acids; estradiol, ethenyl estradiol and other hormones; sphingolipids, ceramides, cholesterol, cholesterol derivatives, phospholipids and other oily ingredients; e-aminocaproic acid, glycyrrhizinic acid, lysozyme chloride, guaiazulene, hydrocortisone, allantoin, tranexamic acid, azulene and other anti-inflammatory agents; vitamins A, B2, B6, C, D, and E, calcium pantothenate, biotin, nicotinic-acid amide, vitamin C ester, and other vitamins.

### [Preferred Parts for Each Components]

The emulsion composition preferably comprises 0.01 to less than 25.0 parts by weight of said (A) a diorganopolysiloxane; 0.01 to less than 20.0 parts by weight of said (B) a softening agent; 8.0 to 60.0 parts by weight of said (C) water; and 20.0 to 90.0 parts by weight of said (D) water-soluble monovalent or polyvalent alcohols. And, the continuous phase of said emulsion composition is an aqueous solution consisting of said (C) water and said (D) water-soluble monovalent or polyvalent alcohols in the ratio of from 1:0.5 to 1:10, and total content of said component (C) and (D) is in the rage of 30.0 and 98.0 parts by weight of said emulsion composition.

As a softening emulsion composition, most preferred continuous phase of this emulsion composition has a glycerin content of from 30 to 90 parts by weight in the total weight pts of emulsion composition. Then, glycerin may be combined with other hypoallergenic alcohols or mixture thereof.

Preferably, the content range of the above component (A) is 4-10 parts by weight, the content range of the above component (B) is 0.5-10 parts by weight, the content range of the above component (C) is 10-40 parts by weight, the content range of the above component (D) is 35-85 parts by weight, the content range of the above component (E) is 9-20 parts by weight, the content range of the above component (F) is 0.05-0.10 parts by weight, the content range of the above component (G) is 0.2-2.5 parts by weight, and the content range of the above component (H) is 0.01-0.3 parts by weight.

### [Fibrous Substrate]

This inventive emulsion composition can be used as a softening emulsion composition for a fibrous substrate, and applied to its fibrous structures. The inventive fibrous substrate treated therewith provides its soft and comfortable feelings in daily use.

“Fibrous structure” as used herein means a structure that comprises one or more fibers, and “Fiber” as used herein means an elongate particulate having an apparent length greatly exceeding its apparent width, i.e. a length to diameter ratio of at least about 10.

“Fibrous substrate” as used herein means a substrate having fibrous structure, and is exemplified by a group of representative fibrous substrates including wipes, fabrics, textiles, and papers. Especially, preferred substrate is a paper for tissue paper products and preferred fiber is a papermaking fiber.

The present invention contemplates the use of a variety of fibers, such as, for example, natural fibers or synthetic fibers, or any other suitable fibers, and any combination thereof. Natural fibers useful in the present invention include animal fibers, mineral fibers, plant fibers and mixtures thereof. Animal fibers may, for example, be selected from the group consisting of: wool, silk and mixtures thereof. Synthetic fibers may, for example, be comprised of cellulose (often referred to as “rayon”); cellulose derivatives such as esters, ether, or nitrous derivatives; polyolefns (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

Plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah and mixtures thereof.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS).

The wood pulp fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Chemy, Elm, Hickory, Poplar, Gum, Walnut, Locust, Sycamore, Beech, Catalpa, Sassafras, Gmelina, Albizia, Anthocephalus, and Magnolia.

Also the present invention are fibers derived from recycled paper, which may contain any or all of the above categories as well as other non-fibrous materials such as fillers and adhesives used to facilitate the original papermaking.

For tissue paper products, the manufacturing process of the fibrous substrate can include known wet-laid papermaking processes and air-laid papermaking processes. Such processes typically include steps of preparing a fiber composition, oftentimes referred to as a fiber slurry in wet-laid processes, either wet or dry, and then depositing a plurality of fibers onto a forming wire or belt such that an embryonic fibrous structure (i.e. an embryonic fibrous web) is formed, drying and/or bonding the fibers together such that a fibrous structure is formed, and/or further processing the fibrous structure such that a finished fibrous structure is formed.

The preferred examples of such tissue papers include conventionally pressed and/or felt-pressed tissue paper; pattern deposited tissue paper; high-bulk, uncompacted tissue paper; creped tissue paper and uncreped tissue paper. These tissue papers may be of a homogenous and/or single layered or multilayered construction; and tissue paper products made therefrom may be of a single-ply or multi-ply construction.

### [Softening Methods and Manufacturing Procedures]

Any known methods for applying the inventive emulsion composition to fibrous substrates can be used in manufacturing procedures. For example, such application method includes spraying, slot extrusion and gravure printing. Other methods include deposition of the emulsion composition onto a forming wire; or fabric or belt which is then contacted by the embryonic fibrous web and/or dried fibrous structure and/or tissue paper product. And also, the inventive emulsion composition can be added into the slurry vats supplying the papermaking machine, during the formation process of the fibrous structure.

The preferred examples of suitable processes for applying the inventive emulsion composition to the fibrous structure include by spraying onto the fibrous structure before it is wound into a roll of paper, by extruding onto the fibrous structure, and by printing onto the fibrous structure and/or tissue paper product. The inventive emulsion composition may be applied to the embryonic fibrous web and/or dried fibrous structure and/or tissue paper product.

The inventive emulsion composition can be applied to the embryonic fibrous web and/or fibrous structure and/or tissue paper product as it is being made on a papermaking machine or thereafter. That is to say, the emulsion can be applied either while it is wet (i.e., prior to final drying) or dry (i.e., after final drying).

Preferably, the inventive emulsion composition can be applied after embryonic fibrous web has been formed. In a typical process, the embryonic fibrous web is formed and then dewatered prior to cationic silicone polymer application in order to reduce the loss of (A) a diorganopolysiloxane or mixtures thereof due to drainage of free water.

Application methods described herein for the inventive emulsion composition can be used with dry or wet embryonic fibrous webs and/or fibrous structures and/or tissue paper product. Among them, one simplest application way is exemplified by immersing the dried tissue paper product in the inventive emulsion composition for a few minutes.

### INDUSTRIAL APPLICABILITY

The emulsion composition of the present invention is useful for the production of fibrous products, providing soft and comfortable feelings to the products treated therewith. The emulsion composition of the present invention is useful especially for the production of tissue paper products, sanitary fibrous products (i.e. sanitary wipes and sanitary tissue papers), cleansing wet wipes, medical and/or cosmetic paper towels, cosmetic puff, female hygiene products, paper diapers, disposable nappy, underwear, fine linens, paper napkins having excellent softness, slippery, moisture/lotion feeling, comfortable fluffiness to human skin and water absorbency.

The inventive fibrous substrates could also be used as other materials for cloths, fabric goods, packing materials, medical materials, kitchen materials, baby-related products, geriatric-care products, pet-accessory products, furniture, building and/or interior materials, stationery supplies and cosmetic products. When the inventive fibrous substrates are used in above-mentioned field, person and/or animals feel soft and comfortable touch to such products, and find excellent functional properties in daily use.

### EXAMPLES

The present invention will be further described in more detail with reference to practical examples and comparative examples, though it is understood that these examples should not be construed as limiting the scope of the present invention.

### [Average Particle Size of Emulsion Particles]

The average particle size of emulsion particles was measured by means of a submicron-particle analyzer (the product of Malvern Instruments Co. Ltd.; Mastersizer 2000) for measuring submicron-size particles by a laser diffraction method.

### [Viscosity of Organpolysiloxanes]

Viscosity of organopolysiloxane was measured by means of a rotary-type viscosimeter of Brookfield Engineering Laboratories. (trademark: Brookfield DV-III ultra type); measurement temperature: 25° C.

[Appearance of the Emulsion Composition]

Appearance of the emulsion composition was evaluated by visual observation. The following criteria were used.

⊚ Completely transparent
◯ Transparent with some turbidity (i.e. semi-transparent or a little hazy)
Δ Somewhat non-uniform and somewhat turbid with milk white

### [Temporal Stability]

The emulsion composition was kept in a quiescent state in a thermostat at room temperature and at 40° C., and then the appearance of the emulsion was observed immediately after preparation and after storage for one month in the thermostat. Criteria for evaluation are shown below.

⊚ No separation of oil phase was observed for more than one year
◯ Slight separation of oil phase was observed
Δ Separation of oil phase was clearly observed
[Hand Feeling Panel Test for Tissue Paper Treated with the Emulsion]

The blank tissue was coated or sprayed with tissue softener emulsion by a coating machine or spraying machine during the rewind process or before cut-fold process.

The hand feeling panel is made up of 10 employees who are experts on sensory evaluation of the product and well trained, not limited to women.

The panel test method is ranking test which is to rank in intensity order (softness, lotion feeling and fluffiness etc) of the presented samples simultaneously. Panelists feel sensory difference and rank them within each group, in each group there are four or three samples grouped. Higher score means better performance. It is necessary to have panel test after aging two weeks when treated tissue reach best performance

### [Panel Test Methodology]

Three samples are presented in a questionnaire for the panelists (10 subjects). The panelists are requested to rank them by performance of its softness, lotion feeling and fluffiness, wherein (mark 4) for “Excellent”, (mark 3) for “Good”, (mark 2) for “Ordinary”, and (mark 1) for “Worse”. Then, we record the average rank of all evaluation as “Hand feeling” results in Tables 1.1 to 1.3. A series of “Hand feeling” results for our working examples and comparative examples are shown in the following tables 1.1 to 1.3. These results show that emulsions of our working examples provide not only softness feeling, but also excellent slippery, moisture/lotion feeling, and comfortable fluffiness to the fibrous substrate treated therewith, compared with those treated with emulsions of comparative examples.

For reference, three raw panel test results are shown in below tables regarding its performance of softness, lotion feeling and fluffiness of tissue papers. In the table, tissue paper (1) is a tissue paper product treated with our inventive emulsions (equivalent to our working example 7), tissue paper (2) is a commercial tissue paper product (as a target reference), tissue paper (3) is raw tissue paper substrate NOT treated with any emulsions (as a blank).

TABLE
Hand Feeling performance for its “softness”
of tissue paper (1) to (3)

| | Tissue Paper | | |
|---|---|---|---|
| Panelists No. | (1) * | (2) | (3) |
| 1 | 4 | 2 | 1 |
| 2 | 4 | 2 | 1 |
| 3 | 4 | 2 | 1 |
| 4 | 4 | 3 | 1 |
| 5 | 3 | 2 | 1 |
| 6 | 3 | 2 | 1 |
| 7 | 3 | 2 | 1 |
| 8 | 4 | 2 | 1 |
| 9 | 4 | 2 | 1 |
| 10 | 3 | 2 | 1 |
| total points | 36 | 21 | 10 |
| Average Record | 4 | 2 | 1 |
| “Softness” | Excellent | Ordinary | Worse |

* Equivalent to our working example 7.

TABLE
Hand Feeling performance for its “lotion
feeling” of tissue paper (1) to (3)

| | Tissue Paper | | |
|---|---|---|---|
| Panelists No. | (1) * | (2) | (3) |
| 1 | 4 | 2 | 1 |
| 2 | 4 | 2 | 1 |
| 3 | 4 | 2 | 1 |
| 4 | 2 | 3 | 1 |
| 5 | 3 | 4 | 1 |
| 6 | 4 | 2 | 1 |
| 7 | 3 | 2 | 1 |
| 8 | 4 | 1 | 2 |
| 9 | 3 | 3 | 1 |
| 10 | 4 | 2 | 1 |
| total points | 35 | 23 | 11 |
| Average Record | 4 | 2 | 1 |
| “lotion feeling” | Excellent | Ordinary | Worse |

* Equivalent to our working example 7.

TABLE
Hand Feeling performance for its “fluffiness”
of tissue paper (1) to (3)

| | Tissue Paper | | |
|---|---|---|---|
| Panelists No. | (1) * | (2) | (3) |
| 1 | 4 | 3 | 1 |
| 2 | 2 | 4 | 1 |
| 3 | 3 | 4 | 1 |
| 4 | 4 | 2 | 1 |
| 5 | 4 | 2 | 1 |
| 6 | 3 | 2 | 1 |
| 7 | 3 | 2 | 1 |
| 8 | 2 | 4 | 1 |
| 9 | 3 | 2 | 1 |
| 10 | 3 | 2 | 1 |
| total points | 31 | 27 | 10 |
| Average Record | 3 | 3 | 1 |
| “lotion feeling” | Good | Good | Worse |

* Equivalent to our working example 7.

### [Water Absorbency]

The water absorbency height or capillary rise of the paper treated with all formulations except the formulations with below Diorganopolysiloxane(A2) passed GB (Chinese standard, GB/T 20808-2006). The water absorbency height or capillary rise on the tested tissue should pass 30 mm above water surface within 100 seconds by the Klemm method

We have other water absorbency test method which is to put a drop of water with 0.01 ml volume from pipette on to the surface of the treated tissue and record the time from start to the water drop be absorbed by the tissue. The time for the formulations without below Diorganopolysiloxane (A2) had no obvious difference.

[(A) A Diorganopolysiloxane]
Diorganopolysiloxane(A1): DiMe siloxane, OH-term. W/aminoethylaminoisobutylmethyl (—CH2CH(CH3)CH2-N(CH2CH(OH)CH2OH)—CH2—NH(CH2CH(OH)CH2OH)) dimethoxysilane, C13-15 alcohols & glycidol
   having 3,000 mPas at 25 degrees C.
Diorganopolysiloxane(A2): Dimethyl siloxane, (ethanediamino-2-methylpropyl)methoxymethylsilyl)oxy- and C13-5-alkoxy-term. having 2,000 mPas at 25 degrees C.
Diorganopolysiloxane(A3): Dimethyl, methyl(3-aminopropyl) siloxane, 3-aminopropylethoxymethylsiloxy-terminated having 1,500 mPas at 25 degrees C.
Diorganopolysiloxane(A4): Dimethyl, Methyl(propyl(poly(EO)(PO))hydroxy) Siloxane, Trimethylsiloxy-terminated having 2,000 mPas at 25 degrees C.

[(B) A Softening Agent]
Softening agent SA 200 (B1): Fatty acid/imidazolidin compound with tallow amido amine (quaternary imidazoline compounds)
Softening agent N100 (B2): A mixture of polyamide resin and poly(oxyethylene) monostearate(C12H25(OCH2CH2)23OH), 20 parts by weight aqueous solution.

[(E) A Moisturizing Agent]
Aloe extract (E1): Aqueous extract from aloe plant.
Sodium Lactate (E2): Sodium lactate aqueous sol. (60. parts by weight)

[(F) Preservatives]
Kathon® LX (F): 5-Chloro-2-methyl-3(2H)-isothiazolone 1.5% aqueous solution (methylisothiazolone-type preservative)

[(G) Surfactants]
Nonionic surfactant (G1): C12H25(OCH2CH2)23OH
Nonionic surfactant (G2): C12H25(OCH2CH2)4OH
Nonionic surfactant (G3): C12H25(OCH2CH2)3OH
Nonionic surfactant (G4): alpha-(C12-C14 sec-Alkyl) omega-Hydroxy Poly(EO)

[(H) pH Adjusting Agents]

Acetic acid (H1), Citric acid (H2) and Phosphoric acid (H3) were used in examples.

### [Preparation Procedure for Working Examples and Comparative Examples]

(B) a softening agent was mixed with a little water into pot, and dispersed by high shear mixer till being homogeneous. Then, (A) a diorganopolysiloxane, (G) surfactants and (H) pH adjusting agents were loaded into above system and dispersed by high shear mixer till being homogeneous. Inversion water is loaded into the above system gradually and mixed the system until the phase inversion happened (emulsification). Dilution water and Glycerin (D) were loaded into the above system, then dispersed by high shear mixer till being homogeneous. Optionally, Kathon® LX (F) and (E) a moisturizing agent are loaded into the system and mixed till getting homogeneous emulsion composition.

The portion of above-mentioned components is shown in the Table 1.1 to 1.3 for all examples.

The average particle size, appearance and temporal stability of each emulsions are shown in the same tables.

Hand feeling panel test results are shown in the same table for tissue paper treated with each emulsions.

Compared with comparative examples 1 to 13, emulsion compositions of our working examples 1 to 8 keep good temporal stability and homogeneous appearance for months in the storage. And, emulsion composition of working examples provide not only softness feeling, but also excellent slippery, moisture/lotion feeling, and comfortable fluffiness to the fibrous substrate treated therewith.

TABLE 1.1
WORKING EXAMPLES

| WORKING Example | | | | | | | | |
|---|---|---|---|---|---|---|---|---|
| *parts by weight | 1 | 2 | 3 | 4 | 5 | 6 | 7 | 8 |
| Diorganopolysiloxane (A1) | 4.37 | 5 | | 4.10 | 4.38 | 4.37 | 4.37 | 4.37 |
| (A2) | | | 9.10 | | | | | |
| (A3) | | | | | | | | |
| (A4) | | | | | 1.28 | 1.28 | 1.28 | 1.28 |
| SA 200 (B1) | 0.57 | 10 | 1.07 | 1.07 | 0.57 | 0.57 | 0.57 | 0.57 |
| N100 (B2) | | | 5.95 | | | | | |
| Nonionic surfactant (G1) | 1.6 | | 2.08 | 0.75 | 1.60 | 1.60 | 1.60 | 1.60 |
| (G2) | 0.32 | | 0.78 | 0.28 | 0.32 | 0.32 | 0.32 | 0.32 |
| (G3) | | | | | | | | |
| (G4) | | 2 | | | | | | |
| Water | 31.43 | 40 | 14.27 | 10.46 | 31.42 | 31.44 | 21.44 | 12.26 |
| Glycerin (D1) | 60.31 | 42.75 | 66.52 | 83.28 | 60.32 | 38.97 | 50.31 | 50.31 |
| Aloe extract (E1) | | | | | | | 20 | 20 |
| Sodium Lactate (E2) | | | | | | 21.34 | | 9.18 |
| Kathon ® LX (F1) | 0.10 | | 0.10 | 0.05 | 0.10 | 0.10 | 0.10 | 0.10 |
| Acetic acid (H1) | | | | | | | | |
| Citric acid (H2) | | | | | | | | |
| Phosphoric acid (H3) | 0.02 | 0.25 | 0.13 | 0.01 | 0.01 | 0.01 | 0.01 | 0.01 |
| Emulsion Properties | | | | | | | | |
| Particle Size (nm) | 143 | 135 | 145 | 423 | 127 | 136 | 142 | 140 |
| Appearance | ⊚ | ⊚ | ⊚ | ◯ | ⊚ | ⊚ | ⊚ | ⊚ |
| Temporal stability | ⊚ | ⊚ | ⊚ | ◯ | ⊚ | ◯ | ⊚ | ◯ |
| Hand feeling | | | | | | | | |
| softness | 4 | 4 | 4 | 4 | 4 | 4 | 4 | 4 |
| lotion feeling | 3 | 2 | 3 | 4 | 3 | 4 | 4 | 4 |
| fluffiness | 3 | 2 | 3 | 3 | 3 | 3 | 3 | 3 |

TABLE 1.2
Comparative EXAMPLES (1)

| Comparative Example | | | | | | | |
|---|---|---|---|---|---|---|---|
| *parts by weight | 1 | 2 | 3 | 4 | 5 | 6 | 7 |
| Diorganopolysiloxane (A1) | | | | | | | |
| (A2) | 13.59 | | 6.82 | 16.94 | 12.45 | 12.45 | 6.22 |
| (A3) | | 13.64 | | | | | |
| (A4) | | | 6.82 | | | | |
| SA 200 (B1) | | | | | | 4.67 | |
| N100 (B2) | 13.59 | 13.64 | 13.64 | 16.94 | 12.45 | 8.71 | 18.67 |
| Nonionic surfactant (G1) | | | | 5.65 | | | |
| (G2) | | | | | | | |
| (G3) | | | | 3.39 | | | |
| (G4) | | | | | | | |
| Water | 63.43 | 54.55 | 54.55 | 56.48 | 66.37 | 65.44 | 66.38 |
| Glycerin (D1) | 9.06 | 18.18 | 18.18 | | 8.30 | 8.30 | 8.30 |
| Aloe extract (E1) | | | | | | | |
| Sodium Lactate (E2) | | | | | | | |
| Kathon ® LX (F1) | | | | | | | |
| Acetic acid (H1) | 0.32 | | | | | | |
| Citric acid (H2) | | | | 0.60 | 0.44 | 0.43 | 0.43 |
| Phosphoric acid (H3) | | | | | | | |
| Emulsion Properties | | | | | | | |
| Particle Size (nm) | 1950 | 2100 | 2510 | 1200 | 4500 | 1220 | 546 |
| Appearance | Δ | Δ | Δ | Δ | Δ | Δ | ◯ |
| Temporal stability | Δ | Δ | Δ | Δ | Δ | Δ | ⊚ |
| Hand feeling | | | | | | | |
| softness | 4 | 4 | 4 | 4 | 4 | 4 | 4 |
| lotion feeling | 1 | 2 | 2 | 1 | 1 | 1 | 1 |
| fluffiness | 4 | 4 | 3 | 3 | 3 | 2 | 3 |

TABLE 1.3
Comparative EXAMPLES (1)

| Comparative Example | | | | | | |
|---|---|---|---|---|---|---|
| *parts by weight | 8 | 9 | 10 | 11 | 12 | 13 |
| Diorganopolysiloxane (A1) | | 7.66 | 7.86 | 6.66 | 4.15 | 4.00 |
| (A2) | 6.22 | | | | | |
| (A3) | | | | | | |
| (A4) | | | | | | |
| SA 200 (B1) | | | | | 10.09 | 10.00 |
| N100 (B2) | 18.67 | 15.33 | 15.73 | 66.58 | | |
| Nonionic surfactant (G1) | | 7.66 | 5.24 | 1.11 | | |
| (G2) | | | | | | |
| (G3) | | | | | | |
| (G4) | | | | | 2.02 | 2 |
| Water | 66.38 | 61.30 | 62.91 | 15.54 | 77.44 | 77.65 |
| Glycerin (D1) | 8.30 | 7.66 | 7.86 | 9.99 | 6.05 | 6.00 |
| Aloe extract (E1) | | | | | | |
| Sodium Lactate (E2) | | | | | | |
| Kathon ® LX (F1) | | | | | | 0.10 |
| Acetic acid (H1) | | | | | | |
| Citric acid (H2) | 0.43 | | 0.39 | | | |
| Phosphoric acid (H3) | | 0.38 | | 0.13 | 0.25 | 0.25 |
| Emulsion Properties | | | | | | |
| Particle Size (nm) | 2700 | 211 | 229 | 133 | 133 | 132 |
| Appearance | Δ | ◯ | ◯ | ◯ | ⊚ | ⊚ |
| Temporal stability | Δ | ⊚ | ⊚ | ⊚ | ⊚ | ⊚ |
| Hand feeling | | | | | | |
| softness | 4 | 4 | 4 | 4 | 4 | 4 |
| lotion feeling | 1 | 1 | 1 | 1 | 1 | 1 |
| fluffiness | 3 | 3 | 3 | 3 | 1 | 1 |

1. An emulsion composition comprising:
(A) 0.01 to less than 25.0 parts by weight of a diorganopolysiloxane;
(B) 0.01 to less than 20.0 parts by weight of a softening agent;
(C) 8.0 to 60.0 parts by weight of water; and
(D) 20.0 to 90.0 parts by weight of water-soluble monovalent or polyvalent alcohols, wherein the continuous phase of said emulsion composition is an aqueous solution consisting of essentially said (C) water and said (D) water-soluble monovalent or polyvalent alcohols in the ratio of from 1:0.5 to 1:10, and total content of said component (C) and (D) is in the rage of 30.0 and 98.0 parts by weight of said emulsion composition.

2. The emulsion composition according to claim 1, wherein said continuous phase of said emulsion composition comprises Glycerin at 30 to 90 parts by weight.

3. The emulsion composition according to claim 1, wherein said component (A) comprises emulsion particles having a mean particle size from 50 to 10,000 nanometers.

4. The emulsion composition according to claim 3, wherein said emulsion particles have of a mean particle size of 50 to 300 nanometers.

5. The emulsion composition according to claim 1, further comprising (E) from 0.01 to less than 20.0 parts by weight of a moisturizing agent.

6. The emulsion composition according to claim 1, wherein said component (A) is a diorganopolysiloxane having at least one group selecting from the group consisting of an amino-functional group, a polyoxyalkylene group, a sulfonate-functional group and an epoxy-functional group in the molecule.

7. A softening emulsion composition for a fibrous substrate comprising the emulsion composition according to claim 1.

8. The softening emulsion composition for a fibrous substrate according to claim 7, wherein said fibrous substrate is selected from the group consisting of wipes, fabrics, textiles, and papers.

9. A method of softening fibrous structures, comprising treating a substrate having fibrous structures with the emulsion composition of claim 1.

10. A fibrous substrate treated with the emulsion composition of claim 1.

11. The fibrous substrate according to 10, wherein said fibrous substrate is a tissue paper product.

12. The emulsion composition according to claim 5, further comprising (F) preservatives.
